(19) United States
(12) Patent Application Publication
(10) Pub. No.: US 2005/0048087 A1
(43) Pub. Date: Mar. 3, 2005

(54) Pharmaceutical compositions for sparingly soluble therapeutic agents

(76) Inventor: Ulrich Posanski, Freiburg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/961,785

(22) Filed: Oct. 8, 2004

Related U.S. Application Data
(63) Continuation of application No. 10/040,842, filed on Jan. 7, 2002, now abandoned.
Application No. 10/040,842 is a continuation of application No. 09/524,965, filed on Mar. 14, 2000, now abandoned.
Application No. 09/524,965 is a continuation of application No. 09/097,915, filed on Jun. 17, 1998, now abandoned.
Application No. 09/097,915 is a continuation of application No. 08/578,527, filed on Jan. 5, 1996, now abandoned.

(30) Foreign Application Priority Data
Jul. 8, 1993 (DE) P 43 22 826.7

(51) Int. Cl. A61K 31/7048; A61K 31/4745; A61K 31/335; A61K 31/19

(52) U.S. Cl. 424/400; 514/291; 514/27; 514/449; 514/570

(57) ABSTRACT

The invention relates to pharmaceutical compositions for sparingly soluble therapeutic agents as well as to a process for the preparation of such compositions. The solubiliser is polyglycerol fatty acid ester or sorbitan fatty acid ester in combination with lipophilic excipients and nonionic surfactants.

The present invention relates to pharmaceutical compositions for sparingly soluble therapeutic agents as well as to processes for the preparation of said compositions.

Generally, the oral administration of a therapeutic agent in solid dosage forms such as tablets, capsules or dragées affords advantages over other, for example parenteral, dosage forms. Diseases that have to be treated by administering injections are felt purely subjectively to be more serious than other diseases in the treatment of which the administration of tablets, capsules or dragées is little noticed. The suitability of such dosage forms for self-medication by patients themselves is especially advantageous, whereas parenteral dosage forms, aside from a few exceptions, have to be administered by the physician or paramedical staff.

After administration and dissolution of an oral dosage form, the gastrointestinal fluid, e.g. gastric or intestinal juice, acts on the therapeutic agents. Many therapeutic agents for oral administration have lipophilic properties and are therefore sparingly soluble in the aqueous environment of the gastrointestinal tract. Under these circumstances, the amount of therapeutic agent which can be resorbed is diminished, resulting in reduced bioavailability. This generally necessitates the application of higher dosages of the therapeutic agent, resulting in biological variability and undesirable variations in efficacy.

To enhance the solubility of sparingly soluble therapeutic agents, so-called solubilisers have been described in the literature, e.g. hydrophilic co-solvents, typically ethanol, propylene glycol, liquid polyethylene glycols, or lipophilic solubilisers, typically lecithin, fatty acid polyglycol ester or fatty acid glycerol polyglycol ester. The use of such solubilisers is problematical owing to reduced tolerance and lack of stability of the dosage form resulting, for example, in dehomogenisation.

Accordingly, DOS 40 05 190 proposes the use of glycerol fatty acid partial esters or partial esters of propylene glycol. The use of these excipients (co-surfactants) is disadvantageous because they are only obtainable in the narrow HLB range from 2 to 3, permitting only limited variation of the ratios of the components present in the carrier composition for adjustment to the different solubilities of the therapeutic agents to be solubilised.

It is the object of this invention to enhance the solubility, resorptive capacity and consequently also the bioavailability of therapeutic agents for oral administration by selecting particularly suitable excipients.

This object is achieved by this invention, which relates to a particularly useful pharmaceutical composition for the enhanced solubilisation of a therapeutic agent which is sparingly soluble in water and present in the carrier composition. The composition of this invention consists of the following components:
a) c. 10-50% by weight, based on the carrier composition, of a co-surfactant which is substantially pure or which is in the form of a mixture, having a hydrophilic-lipophilic balance of less than 10 (HLB value according to Griffin), selected from the group consisting of polyglycerol fatty acid esters and sorbitan fatty acid esters;
b) c. 5-40% by weight, based on the carrier composition, of a pharmaceutically acceptable oil which is substantially pure or which is in the form of a mixture, comprising a triglyceride as essential lipophilic component; and
c) c. 10-50% by weight, based of the carrier composition, of a nonionic surfactant which is substantially pure or which is in the form of a mixture, having a HLB value of more than 10;
   and further optional pharmaceutically acceptable excipients.

The invention also relates to the process for the preparation of a pharmaceutical composition containing a solubilised therapeutic agent which is sparingly soluble in water and present in a carrier composition comprising the indicated components. This pharmaceutical composition is suitable for filling into oral dosage units, e.g. into starch or hard or soft gelatine capsules.

Within the scope of the description of this invention, the terms used above and hereinafter are defined as follows:

The term “pharmaceutical composition” defines the mixture of a solubilised pharmaceutical therapeutic agent, or a combination of therapeutic agents, which is sparingly soluble in water and present in a carrier composition comprising the indicated components, which mixture can be processed to oral dosage forms, preferably starch or hard or soft gelatine capsules.

The term “solubilised” or “solubilisation” of a therapeutic agent or therapeutic agent mixture which is sparingly soluble in water defines a dispersion process induced by the action of a suitable solubiliser which enhances the dispersibility of the therapeutic agent to such a degree that a therapeutically effective dosage is completely dissolved or made at least bioavailable by a partial dissolution process. The term “dispersibility” defines a measure for the formation of micro-emulsions, of genuine molecular solutions of the therapeutic agents and the excipients in water, and of colloidal solutions, typically solutions of association colloids or molecular colloids which are clear or opalescent, and which contain no solid particles at all after optional filtration, preferably with sterile filters having a pore diameter of c. 5-10 μm, or of e.g. micellar solutions or spherocolloids which can only be separated in an ultracentrifuge. The dispersibility can be given, for example, in mg or mmol per litre of water.

A therapeutic agent or therapeutic agent mixture which is sparingly soluble in water has a solubility in water of less than 500 mg/1000 ml, preferably of less than 200 mg/ml.

Particularly suitable sparingly soluble therapeutic agents are immunosuppressants having a macrolide structure, typically cyclosporin A, cyclosporin G, rapamycin, tacrolimus, deoxyspergualin, mycophenolate-mofetil, gusperimus, non-steroidal antiphlogistic agents, typically acetylsalicylic acid, ibuprofen or S(+)-ibuprofen, indomethacin, diclofenac, piroxicam, meloxicam, tenoxicam, naproxen, ketoprofen, flurbiprofen, fenoprofen, felbinac, sulindac, etodolac, oxyphenbutazone, phenylbutazone, nabumetone; dihydropyridine derivatives having cardiovascular activity, e.g. nifedipine, nitrendipine, nimodipine, nisoldipine, isradipine, felodipine, amlodipine, nilvadipine, lacidipine, benidipine, masnidipine, furnidipine, niguldipine; depressants and stimulants, typically α-liponic acid, muramyl peptides, e.g. muramyl dipeptide or muramyl tripeptide, romurtid, fat-soluble vitamins, typically vitamin A, D, E or F; alkaloids, e.g. vincopectin, vincristine, vinblastin, reserpine, codeine, ergot alkaloids, typically bromocriptine, dihydroergotamine, dihydroergocristine; antitumour agents, e.g. chlorambucil, etoposide, teniposide, idoxifen, tallimustin, teloxantron, tirapazamine, carzelesin, dexniguldipine, intoplicin, idarubicin, miltefosin, trofosfamide, teloxantrone, melphalan, lomustine, 4,5-bis(4′fluoroanilino)phthalimide; 4,5-dianilinophthalimide; immunomodulators, typically thymoctonan, prezatid copper acetate; antiinfectives, e.g. erythromycin, daunorubicin, gramicidin, doxorubicin, amphotericin B, gentamycin, leucomycin, streptomycin, ganefromycin, rifamexil, ramoplanin, spiramycin; antimycotic agents, typically fluconazole, ketoconazole, itraconazole; H2-receptor antagonists, typically famotidine, cimetidine, ranitidine, roxatidine, nizatidine, omeprazole, proteinkinase inhibitors, e.g. N-[4-methyl-3-(4-pyridin-3-ylpyrimidin-2-ylamino)phenyl]benzamide, N-benzoylstaurosporin; HIV-1-protease inhibitors, e.g. BOC-Phe^{c}Phe-Val-Phe-morpholine or its O-[2-(2-methoxyethoxy)acetoxy] derivative; leucotriene antagonists, typically N-[4-(5-cyclopentyloxycarbonylamino-1-methylindol-3-ylmethyl)-3-methoxybenzoyl]-2-vinyloxy]benzenesulfonamide.

Particularly preferred therapeutic agents are cyclosporins, rapamycin, tacrolimus, deoxyspergualin, mycophenolate-mofetil, nifedipine, nimodipine, etoposide, ibuprofen and α-liponic acid.

Instead of being in the form of a free acid or in basic form, the therapeutic agent may be present in the pharmaceutical composition in the form of a pharmaceutically acceptable salt, typically as hydrobromide, hydrochloride, mesylate, acetate, succinate, lactate, tartrate, fumarate, sulfate, maleate, and the like.

The concentration of the therapeutic agent or combination thereof is determined by the dosage to be administered and can be in the range from 1 to 30% by weight, preferably from 5 to 20% by weight, more particularly from 5 to 12% by weight, based on the weight of the carrier composition.

The carrier composition for one of the cited therapeutic agents or for a therapeutic agent combination is defined as follows:

The requirement “substantially pure” with respect to a component present in the carrier composition defines a degree of purity higher than 90%, preferably higher than 95%, of this component, prior to being mixed with the other components of the therapeutic agent combination. A component defined as “substantially pure” preferably has a uniformly defined structure and composition.

Components present as mixture in the carrier composition can be mixtures of natural substances whose composition depends on the raw material itself, on its isolation and its further processing. The components of such mixtures are indicated in the specifications of the producer.

The polyglycerol fatty acid ester of component a) consists of a substantially pure polyglycerol fatty acid ester or of a mixture of different polyglycerol fatty acid esters, wherein the polyglycerol chain preferably contains up to and including 10 units of glycerol which are esterified with 1-10 acid radicals of saturated or unsaturated carboxylic acids having an even number of 8-20 carbon atoms.

The acid radical of a saturated carboxylic acid having an even number of 8-20 carbon atoms which esterifies the polyglycerol chain is preferably straight-chain and contains 12, 14, 16 and 18 carbon atoms, typically n-dodecanoyl, n-tetradecanoyl, n-hexadecanoyl or n-octadecanoyl.

The acid radical of an unsaturated carboxylic acid having an even number of 8-20 carbon atoms, which esterifies the polyglycerol chain, is preferably straight-chain and contains 12, 14, 16 and 18 carbon atoms and 1 double bond, typically 9-cis-dodecenoyl, 9-cis-tetradecenoyl, 9-cis-hexadecenoyl or 9-cis-octadecenoyl.

The following names are also conventionally used for the cited acid radicals: 9-cis-dodecenoyl(lauroleoyl), 9-cis-tetradecenoyl(myristoleoyl), 9-cis-hexadecenoyl (palmitoleoyl), 6-cis-octadecenoyl(petroseloyl), 6-trans-octadecenoyl(petroselaidoyl), 9-cis-octadecenoyl(oleoyl), 9-trans-octadecenoyl(elaidoyl), 11-cis-octadecenoyl (vaccenoyl), 9-cis-icosenoyl(gadoleoyl), n-dodecanoyl(lauroyl), n-tetradecanoyl (myristoyl), n-hexadecanoyl(palmitoyl), n-octadecanoyl(stearoyl), n-icosanoyl (arachidoyl).

Suitable polyglycerol fatty acid esters having a uniformly defined structure are typically diglycerol monocaprate, diglyceryl monolaurate, diglycerol diisostearate, diglycerol monoisostearate, diglycerol tetrastearate(polyglyceryl 2-tetrastearate), triglycerol monooleate(polyglyceryl 3-monooleate), triglycerol monolaurate, triglycerol monostearate(polyglyceryl 3-stearate), triglycerol monoisosterate, hexaglycerol dioleate(polyglycerol 6-dioleate), hexaglycerol distearate(polyglycerol 6-distearate), decaglycerol dioleate(polyglycerol 10-dioleate), decaglycerol tetraoleate(polyglycerol 10-tetraoleate), decaglycerol decaoleate(polyglycerol 10-decaoleate), decaglycerol decastearate(polyglycerol 10-decastearate). The CTFA nomenclature is given within the brackets. These products are commercially available under the registered trade mark Caprol® (trade mark of Karlshamns USA Inc., Columbus Ohio). Specific product names: CAPROL 2G4S, 3GO, 3GS, 6G2O, 6G2S, 10G2O, 10G4O, 10G10O, 10G10S. Further products are available under the names of DGLC-MC, DGLC-ML, DGLC-DISOS, DGLC-MISOS, TGLC-ML and TGLC-MISOS from Solvay Alkali GmbH, D-3002 Hannover.

The mixture of different polyglycerol fatty acid esters is specified under names such as decaglycerol monooleate, dioleate, polyglycerol ester of mixed fatty acids, polyglycerol ester of the fatty acids, polyglycerol caprate, cocoate, laurate, lanolinate, isostearate or rizinolate and are commercially available under the registered trade mark Triodan® and Homodan® (trade mark of Grindsted Products, Grindsted Denmark), specific product names: TRIODAN 20, 55, R90 and HOMODAN MO; Radiamuls® (trade mark of Petrofina (FINA), Bruxelles Belgium), specific product name: RADIAMULS Poly 2253; under the name CAPROL PGE 860 or ET, or under the registered trade mark Plurol® (trade mark of Gattefossé Etablissements, Saint-Priest, France), specific product name: PLUROL Stearique WL 1009 or PLUROL Oleique WL 1173. Further products are available under the names PGLC-C 1010 S, PGLC-C 0810, PGLC 1010/S, PGLC-L T 2010, PGLC-LAN 0510/S, PGLC-CT 2010/90, PGLC-ISOS T UE, PGLC-R UE, PGLC-ISOS 0410 from Solvay Alkali GmbH, D-3002 Hannover.

The cited polyglycerol fatty acid esters conform to the specifications listed in the Foodchemical Codex FCC III under “Monographs”, p.232 regarding “description”, “requirements” and “tests”. Applicable are especially the product specifications published by the indicated producers on the data sheets of the specified product, in particular specifications such as monoester content, drop point, free glycerol, free fatty acid, iodine value, form, antioxidants, HLB value, properties and stability.

The cited polyglycerol fatty acid esters in particular conform to the requirements of number E 475 of the EC food additives directive (EC directive 74/329) as well as the regulation of U.S. FDA Code 21 CFR §172.854.

The sorbitan fatty acid ester of component a) preferably consists of a sorbitan fatty acid ester which is substantially pure, or of a mixture of different sorbitan fatty acid esters, and the sorbitan skeleton is esterified with 1-3 acid radicals of a saturated or unsaturated straight-chain carboxylic acid having an even number of 8-20 carbon atoms.

The acid radical of a saturated carboxylic acid having an even number of 8-20 carbon atoms which esterifies the sorbitan skeleton is preferably straight-chain with 12, 14, 16 and 18 carbon atoms, typically n-dodecanyol, n-tetradecanoyl, n-hexadecanoyl or n-octadecanoyl.

The acid radical of an unsaturated carboxylic acid having an even number of 8-20 carbon atoms is preferably straight-chain with 12, 14, 16 and 18 carbon atoms, typically oleoyl.

Suitable sorbitan fatty acid esters are preferably sorbitan monolaurate, sorbitan monopalmitate, sorbitan monostearate, sorbitan tristearate, sorbitan monooleate, sorbitan sesquioleate and sorbitan trioleate. These products are commercially available under the registered trade mark Span® (trade mark of Atlas, Wilmington USA), specific product names: SPAN 20, 40, 60, 65, 80 and 85; Arlacel® (trade mark of Atlas), specific product names: ARLACEL 20, 40, 60, 80, 83, 85 and C; Crill® (trade mark of Croda Chemicals Ltd., Cowick Hall, Snaith Goole GB), specific product names: CRILL 1, 3 and 4; Dehymuls® (trade mark of Henkel, Düsseldorf DE), specific product names: DEHYMULS SML, SMO, SMS, SSO; Famodan® (trade mark of Grindsted Products, Grindsted Denmark), specific product names: FAMODAN MS and TS; Capmul® (trade mark of Karlshamns USA Inc., Columbus Ohio), specific product names: CAPMUL S and O; Radiasurf® (trade mark of Petrofina (FINA), Bruxelles Belgium), specific product names: RADIASURF 7125, 7135, 7145 and 7155.

The cited sorbitan fatty acid esters and the polyglycerol fatty acid esters conform to the specifications listed in the British Pharmacopeia (special monography) or in Ph. Helv. VI. Applicable are especially the product specifications published by the indicated producers on the data sheets of the specified product, in particular specifications regarding e.g. form, colour, HLB value, viscosity, ascending melting point and solubility.

Component a) has a HLB value of less than 10. Component a) is present in the carrier composition in an amount of 10-50% by weight, preferably 15-40% by weight, more particularly 15-20% by weight, based on the total weight of the carrier composition. Component a) can also consist of product mixtures of the cited polyglycerol fatty acid esters with each other or of the cited sorbitan fatty acid esters with each other, or of product mixtures of said polyglycerol fatty acid esters with said sorbitan fatty acid esters.

A pharmaceutically acceptable oil b) is a triglyceride of natural origin or a synthetic or semi-synthetic substantially pure triglyceride. It is preferred to use a triglyceride of natural origin wherein the glycerol is esterified by acid radicals of saturated or unsaturated carboxylic acids having an even number of 8-20 carbon atoms. Such acid radicals are defined above and are typically n-dodecanoyl, n-tetradecanoyl, n-hexadecanoyl, n-octadecanoyl or oleoyl.

Suitable triglycerides of natural orgin are, for example, ground nut oil, sesame oil, sunflower oil, olive oil, corn oil, soybean oil, castor oil, cottonseed oil, rape-seed oil, thistle oil, grape-seed oil, fish oil or neutral oil.

Component b) is present in the carrier composition in an amount of c. 5-40% by weight, preferably 10-35% by weight, based on the total weight of the carrier composition. Component b) can also consist of product mixtures of the indicated pharmaceutically acceptable oils.

The nonionic surfactant of component c) having a HLB value of more than 10 is preferably an amphiphilic substance whose hydrophilic component consists of polyethylene oxide, the average molecular weight of the polyethylene oxide component being c. 600-2500, corresponding to 15-60 units of ethylene oxide.

Suitable nonionic surfactants are typically reaction products of natural or hydrogenated castor oil and ethylene oxide. Such products are commercially available, e.g. under the registered trade mark Cremophor®, Niccol® and Emulgin®. Suitable nonionic surfactants are also polyoxyethylene (POE) sorbitan fatty acid esters (polysorbates), typically POE-(20)sorbitan monolaurate, POE-(20)sorbitan monopalmitate, POE-(20)sorbitan tristearate, POE-(20)sorbitan monooleate or POE-(20)sorbitan trioleate as well as polyoxyethylene fatty acid esters, typically POE-(20, 30, 40, 50)stearate. Such products are commercially available e.g. under the registered trade marks Tween® and Myrj®.

Component c) is present in the carrier composition in an amount of c. 10-50% by weight, preferably 20-45% by weight, based on the total weight of the carrier composition. Component c) can also consist of product mixtures of the indicated pharmaceutically acceptable nonionic surfactants.

Suitable pharmaceutically acceptable additional excipients are added to the carrier composition in such an amount as to make up 100% by weight together with the amounts of components a), b) and c) as well as of the therapeutic agent or combination thereof. Additional excipients can be present in the carrier composition in amounts of 0% to c.75% by weight. Additional excipients depend on the choice of the pharmaceutical dosage form. Pharmaceutically acceptable diluents are added to liquid dosage forms, such as drops, suspensions or capsule fillings, typically ethanol, propanol, isopropanol, propylene glycol, polyethylene glycol, glycerol or water, or mixtures thereof.

Conventional excipients can also be added, for example preservatives, typically benzyl alcohol, ethanol, p-hydroxybenzoate, sorbic acid; antioxidants, typically tocopherols, butylhydroxyanisol, butylhydroxytoluene, ascorbic acid, ascorbylpalmitate; stabilisers, typically citric acid, tartaric acid, EDTA, flavourings or fragrances.

Gelatin capsules are suitably filled with conventional plasticisers to stabilise the gelatin shell. Such excipients are typically sorbitol, sorbitan, polyvinylpyrrolidone, hydroxypropylmethyl cellulose (HPMC), hydroxypropyl cellulose, methyl cellulose or colloidal silicon dioxide.

The invention also relates to the process for the preparation of the above-defined pharmaceutical composition, which comprises mixing components a), b) and c) and optional further pharmaceutically acceptable excipients in any order, dispersing in this mixture the pharmaceutical therapeutic agent which is sparingly soluble in water and, if desired, processing the dispersion to a suitable dosage form for oral administration.

Dispersion of the therapeutic agent or therapeutic agent combination can be carried out after blending components a), b) and c) and the other excipients. Alternatively, the therapeutic agent or therapeutic agent combination can be dispersed in a single component or in a mixture of two of the indicated components, and the remaining components can then be added. Solubilisation or dispersion processes can be accelerated by heating single components or mixtures thereof. Preferred reaction conditions are those promoting the formation of a colloidally dispersed phase.

The process is carried out in an inert gas atmosphere, typically under nitrogen, helium or argon, in the presence of therapeutic agents susceptible to oxygen.

Before carrying out said process, the oxygen present in the liquid components can be removed by application of low pressure, typically of 50-100 mbar, or by ultrasonication. This process is suitably carried out using a double-walled reaction vessel equipped with stirrer.

The conversion into a dosage form for oral administration is carried out in per se known manner. Dosage forms for oral administration, such as drops, suspensions, emulsions and the like, can be prepared by conventional methods described in standard text books such as in Hagers Handbuch der Pharmazeutischen Praxis or Remington's Pharmaceutical Sciences.

Capsules are preferably dry-filled capsules made of gelatin and, in some cases, with the addition of glycerol or sorbitol, and which dissolve without delay under the action of gastric juice. Alternatively, capsules made of starch can be used, e.g. those available under the registered trade mark Capill®, supplied by Capsugel/Warner Lambert. The capsules may be blended with further excipients and fillers, typically lactose, starch, lubricants, e.g. starch or magnesium stearate. Soft capsules can additionally contain liquids such as lecithin, fats, oils, paraffin oil or liquid polyethylene glycol. Depending on the dosage, dry-filled capsules are suitably of size 0-4 and, preferably, of size 0-2. Suitable commercially available capsules are those supplied by Shionogi, Capsugel or Scherer.

The following Examples illustrate the invention in more detail without restricting the general scope defined above. The cited therapeutic agents are representative of all the therapeutic agents indicated above. Temperatures are given in degrees centigrade.

### EXAMPLE 1

Composition for filling into soft gelatin capsules; amounts in mg per filled capsule; size of soft gelatin capsules: 22 minims oblong.

| | | |
|---|---|---|
| 1 | Ciclosporin A (USP XXII/Pharm.Eur.) | 100.0 |
| 2 | POE-(40) hydrogenated castor oil | 400.0 |
| | (CREMOPHOR RH 40, NICCOL | |
| | HCO 40, SIMULSOL 1293) | |
| 3 | Di/tri/tetraglycerol fatty acid ester | 238.0 |
| | (FCC/TRIODAN 20) | |
| 4 | Sesame oil (DAB 10) | 160.0 |
| 5 | alpha-Tocopherol (DAB 10) | 2.0 |
| 6 | Ethanol (DAB 10) | 100.0 |

Components 2-4 are mixed in a stainless steel vessel equipped with stirrer, while heating to 40°. The solution is then degassed by applying low pressure. Antioxidant 5 is added to the clear solution, and the therapeutic agent ciclosporin A is then dispersed therein. After addition of the ethanol, the entire composition is stirred until a clear solution is obtained. This solution is cooled to c. 20° and then filled into soft gelatin capsules. To compensate for evaporation, the amount of ethanol added is 30-60 mg higher than in the above composition.

In addition to gelatin, the shells of the soft gelatin capsules contain excipients which influence the consistency, typically glycerol and/or propylene glycol, or sorbitol and/or mannitol. The shells can additionally contain pigments or colourants, typically titanium dioxide, iron oxide, quinoline yellow, or cochenille red A.

### EXAMPLE 2

Composition for filling into hard gelatin capsules or starch capsules; amounts in kg per preparation.

| | | |
|---|---|---|
| 1 | Nifedipine (DAB 10) | 20.0 |
| 2 | POE-(20) sorbitan monooleate | 168.0 |
| | (Polysorbate 20 Pharm.Eur., TWEEN 20) | |
| 3 | Triglycerol mono/dioleate (FCC - CAPROL 3GO) | 28.0 |
| 4 | Neutral oil (MIGLYOL 812, CAPTEX 300/400) | 84.0 |

All components of the composition are mixed at 45° in a double-walled heating vessel having a volume of 300 l and are stirred until a clear solution is obtained. 300 mg each of the cooled clear solution are filled into hard gelatin capsules of size 1 made opaque with titanium dioxide/iron oxide.

The filled capsules are banded. Owing to the susceptibility of nifedipine to light, all process steps must be carried out excluding daylight.

### EXAMPLE 3

Composition for filling into glass bottles. The composition is suitable for oral administration as drop solution and is filled into a brown 40 ml dropping bottle. Amounts are given in gram.

| | | |
|---|---|---|
| 1 | Nimodipine | 3.0 |
| 2 | POE-(60) hydrogenated castor oil | 15.0 |
| | (CREMOPHOR RH 60, NICCOL | |
| | HCO 60, SIMULSOL 1294) | |
| 3 | Sorbitan monolaurate (BPC 1973, SPAN 20) | 8.5 |
| 4 | Sunflower oil (DAB 10) | 8.5 |
| 5 | Propylene glycol | 5.0 |

### EXAMPLE 4

Composition for filling into soft gelatin capsules; amounts in mg per filled capsule; size of soft gelatin capsule: 4 minims oblong.

| | | |
|---|---|---|
| 1 | Tacrolimus | 10.0 |
| 2 | POE-(35) castor oil (CREMOPHOR EL) | 72.0 |
| 3 | Sorbitan monooleate (SPAN 80) | 72.0 |
| 4 | Neutral oil | 32.0 |
| 5 | alpha-Tocopherol | 1.0 |
| 6 | Propylene glycol (DAB 10) | 5.0 |

The capsules are prepared in general accordance with the procedure of Example 1. Propylene glycol is particularly suitable as plasticiser for the capsule shell.

### EXAMPLE 5

Composition for filling into hard gelatin capsules; amounts relate to the filling of one size 0 capsule.

| | | |
|---|---|---|
| 1 | alpha-Liponic acid | 100.0 |
| 2 | POE-(40) stearate (US/NF, MYRJ 52 S) | 80.0 |
| 3 | Tetraglycol stearate (FCC, TRIODAN 55) | 215.0 |
| 4 | Sesame oil | 160.0 |
| 5 | Butylhydroxyanisol | 0.5 |

The solution is prepared in general accordance with the procedure of Example 2, additionally observing the susceptibility of the liponic acid to oxygen.

### EXAMPLE 6

Composition for filling into soft gelatin capsules; Amounts in mg per filled capsule, Size of soft gelatin capsules: 6 minims, oblong.

| | | |
|---|---|---|
| 1 | Rapamycin | 20.0 |
| 2 | POLYSORBAT 80 (TWEEN 80) | 150.0 |
| 3 | Sorbitan monoleate | 25.0 |
| 4 | Neutral oil | 75.0 |
| 5 | Ascorbylpalmitate | 0.5 |
| 6 | Benzyl alcohol (DAB 10) | 5.0 |

The composition is prepared in general accordance with the procedure of Example 1, adding the benzyl alcohol as last component.

### EXAMPLE 7

Composition for filling into soft gelatin capsules; amounts in mg per filled capsule.

| | | |
|---|---|---|
| 1 | Etoposide | 100.0 |
| 2 | POE-(40) hydrogenated castor oil | 400.0 |
| 3 | Di/tri/tetraglycerol laurate | 160.0 |
| | (TGLC-Laurat T2010 Solvay Alkali GmbH) | |
| 4 | Corn oil | 230.0 |
| 5 | Ethanol | 100.0 |

The composition is prepared in general accordance with the procedure of Example 1.

### EXAMPLE 8

Composition for use in soft gelatin capsules; amounts in mg per filled capsule; size of soft gelatin capsule: 9.5 minims, oblong.

| | | |
|---|---|---|
| 1 | S(+)-Ibuprofen | 100.0 |
| 2 | POLYSORBAT 60 (TWEEN 60) | 210.0 |
| 3 | Hexaglycerol dioleate (CAPROL 6G2O) | 130.0 |
| 4 | Castor oil (DAB 10) | 60.0 |

The composition is prepared in general accordance with the procedure of Example 1.

1-10. (Canceled)

11. A pharmaceutical composition comprising a solubilized therapeutic agent which is cyclosporin A or cyclosporin G and a carrier composition, said carrier composition comprising:
a) about 10-50% by weight, based on the carrier composition, of a polyglycerol fatty acid ester co-surfactant which is substantially pure or which is in the form of a mixture, having a hydrophilic-lipophilic balance of less than 10 (HLB value according to Griffin);
b) about 5-40% by weight, based on the carrier composition, of a pharmaceutically acceptable oil which is substantially pure or which is in the form of a mixture, comprising a triglyceride as essential lipophilic component; and
c) about 10-50% by weight, based on the carrier composition, of a nonionic surfactant which is substantially pure or which is in the form of a mixture, having an HLB value of more than 10;
and further optional pharmaceutically acceptable excipients.

12. A pharmaceutical composition of claim 11, comprising about 1-30% by weight, based on the total weight of the carrier composition, the therapeutic agent having a solubility in pure water of less than 500 mg/1000 mL.

13. A pharmaceutical composition of claim 11, wherein the therapeutic agent is cyclosporin A.

14. A pharmaceutical composition of claim 11, wherein the therapeutic agent is cyclosporin G.

15. A pharmaceutical composition of claim 11, wherein the polyglycerol chain contains up to and including 10 units of glycerol which are esterified with 1-10 acid esters of saturated or unsaturated carboxylic acids having an even number of 8-20 carbon atoms.

16. A pharmaceutical composition of claim 11, wherein component a) contains as polyglycerol fatty acid substantially pure polyglyceryl 2-tetrastearate, polyglyceryl 3-monooleate, polyglyceryl 3-stearate, polyglyceryl 6-dioleate, polyglyceryl 6-distearate, polyglyceryl 10-dioleate, polyglycerly 10-tetraoleate, polyglyceryl 10-decaoleate or polyglyceryl 10-decasterate, or a mixture of these compounds.

17. A pharmaceutical composition of claim 11, wherein
component b) contains as pharmaceutically acceptable oil ground nut oil, sesame oil, sunflower oil, olive oil, corn oil, soybean oil, castor oil, cottonseed oil, rapeseed oil, thistle oil, grapeseed oil, fish oil or neutral oil; and
component c) contains a nonionic surfactant with a hydrophilic component consisting of 15-60 units of ethylene oxide.

18. A process for the preparation of a pharmaceutical composition of claim 11, which comprises mixing components a), b), and c) and further optional pharmaceutically acceptable water-soluble excipients in any order, dispersing in this mixture the therapeutic agent and, if desired, processing the dispersion to a suitable dosage form for oral administration.

19. A process of claim 18, which comprises filling the dispersion into starch or hard or soft gelatin capsules.
